SEQUENCE LISTING (60 sequences)

SEQ ID NO: 1 - protein, 115 aa, Glycine max
MEGSSPSIEEERTATFYVYHPCYFLQQALRALLKCVGIDESENTMCSQANKQEKSSLPQTPSADDPITNSPTHKSSPDAADPPSTTNQTIIIASLMATRGSRGSKISDGSGPQHN

SEQ ID NO: 2 - protein, 118 aa, Glycine max
MEGSSASSHEEERTATFYVYHPCYFLQQAFRALLRCLGIESEATMCSKAEEEKSSLSQTTAADDLITNSPSCNISHKNSQDAADPPSTTNQTIIIASSMARRGNRGSRISHGSGPQHN

SEQ ID NO: 3 - protein, 94 aa, Glycine max
MPRLLGNLTPQILVQALRSMPRQATSSLKVRTHGGTRGSGPSHGSVGGKRGSPISQGKGGQHNEVVQKMPNGNNGSKRNDEARVEANAAKKLGN

SEQ ID NO: 4 - protein, 171 aa, Zea mays
MDEHGEKEENKSQDSALAAEQREETAAAEGEDTSEESTDQREDGHGYKADESAGLLPEDDVGSGEASAAPHFGHPCSLLRACAGFLGLHGCGGDQKPAAAAVAASAAEAATAAAASSSQDEEDGGVDMAKANGFYMHEVITRVWAVAVRRPRPRPPDHAREGSGGNGGVHH

SEQ ID NO: 5 - protein, 142 aa, Zea mays
MDERGEKEEEHGVVEEETAAVVLKEVEVEMEMVGGSEEASAAPLLLAHPCSLLQLLLRACAGCLVRLLHGHCSDGANDDPKAAADDDDAAPEAAAAAAAAAGDGGDKAATYLYMQEVWAVRRRPTTPGRPREGSGGNGGNHH

SEQ ID NO: 6 - protein, 199 aa, Zea mays
MALSPSAQATSPLARQLLRHVSSGLVAAAFHRPAPAISLPSAPRGADDVALAASHHQRLWPAPSPKGRPGAPRQGSGGQVHHAAPAMATIASAVMLPTNAVTAAAVTTHVVTRGPARPGLPAQGGGGKTHGAPAAAKARAALRGPAPPARPKEGSGGKVHVVSPAAMSSASVLMRGPAPPGHPAEGAGGRGGSIHAISS

SEQ ID NO: 7 - protein, 23 aa, Glycine Max
ASLMATRGSRGSKISDGSGPQHN

SEQ ID NO: 8 - protein, 23 aa, Glycine max
ASSMARRGNRGSRISHGSGPQHN

SEQ ID NO: 9 - protein, 23 aa, Glycine max
PSHGSVGGKRGSPISQGKGGQHN

SEQ ID NO: 10 - protein, 54 aa, Glycine max
PSHGSVGGKRGSPISQGKGGQHNEVVQKMPNGNNGSKRNDEARVEANAAKKLGN

SEQ ID NO: 11 - protein, 23 aa, Zea mays
VRRRPTTPGRPREGSGGNGGNHH

SEQ ID NO: 12 - protein, 23 aa, Zea mays
RRPRPRPPDHAREGSGGNGGVHH

SEQ ID NO: 13 - protein, 27 aa, Zea mays
LMRGPAPPGHPAEGAGGRGGSIHAISS

SEQ ID NO: 14 - protein, 23 aa, Arabidopsis thaliana
QTPTGRKGKEDKPVREGPPPQHN

SEQ ID NO: 15 - protein, 28 aa, Arabidopsis thaliana
QTPTGRKGKEDKPVREGPPPQHNQRWEG

SEQ ID NO: 16 - protein, 23 aa, Brassica napus
GSKVKAKKKDKEKVSSGRPGQHH

SEQ ID NO: 17 - protein, 23 aa, Gossypium hirsutum
RINLMGYDYSGYGQSSGKPSEHN

SEQ ID NO: 18 - protein, 49 aa, Gossypium hirsutum
RINLMGYDYSGYGQSSGKPSEHNTYADIEAAYKCLEESYGAKQENIILY

SEQ ID NO: 19 - protein, 23 aa, Oryza sativa
ADSAPQRPGAPAEGAGGNGGAVH

SEQ ID NO: 20 - protein, 32 aa, Oryza sativa
ADSAPQRPGAPAEGAGGNGGAVHVAPAAAASS

SEQ ID NO: 21 - protein, 23 aa, Oryza sativa
ADSAPQRPGAPAEGAGGNGGDVH

SEQ ID NO: 22 - protein, 32 aa, Oryza sativa
ADSAPQRPGAPAEGAGGNGGDVHVAPAAATSS

SEQ ID NO: 23 - protein, 23 aa, Oryza sativa
ADSAPQRPGSPAEGAGGNGGAVH

SEQ ID NO: 24 - protein, 35 aa, Oryza sativa
ADSAPQRPGSPAEGAGGNGGAVHAAPAAAAAAASS

SEQ ID NO: 25 - protein, 23 aa, Oryza sativa
SKPKPEPPGYPREGGGGNGGVVD

SEQ ID NO: 26 - protein, 42 aa, Oryza sativa
SKPKPEPPGYPREGGGGNGGVVDDVSPSSTDTSTSSSSSSSS

SEQ ID NO: 27 - protein, 22 aa, Oryza sativa
AMPRSERPVLREGNGGKGGAHH

SEQ ID NO: 28 - protein, 26 aa, Oryza sativa
AMPRSERPVLREGNGGKGGAHHNAGL

SEQ ID NO: 29 - protein, 23 aa, Triticum aestivum
RQLAGPKRPDPREGRGGGGGAIH

SEQ ID NO: 30 - protein, 27 aa, Triticum aestivum
RQLAGPKRPDPREGRGGGGGAIHAFSS

SEQ ID NO: 31 - DNA, 348 bp, Glycine max
atggaagggtcttcaccatccattgaagaagagagaacagccactttctatgtgtaccatccttgctattttcttcaacaagcactcagggctctcttgaagtgtgtaggtattgatgagtctgaaaacacaatgtgttcacaggccaataaacaagagaaaagctcactgccacaaactccttctgcagatgatcctattacaaactctccaacccacaaaagctccccagatgctgcagatccaccttccacaactaatcaaaccattatcattgcaagtttaatggcaacgcgtggcagtcgagggtctaaaattagcgatgggtcaggccctcagcataattaa

SEQ ID NO: 32 - DNA, 357 bp, Glycine max
atggaagggtcttcagcatcatcgcatgaagaagagagaacagccactttctatgtgtaccatccttgctattttcttcaacaagcattcagggctctcttgaggtgtctaggtattgagtctgaagccacaatgtgttcaaaggcagaagaagagaaaagctcactgtcacaaactactgctgcagatgatcttattacaaactctccaagctgcaacatatcccacaaaaactcccaagatgctgcagatccaccatccacaactaatcaaaccattatcattgcaagttcaatggcaaggcgtggaaatcgagggtctagaattagccatgggtcaggccctcagcataattaa

SEQ ID NO: 33 - DNA, 429 bp, Zea mays
atggatgagcgcggggagaaggaggaggagcacggagtagtggaggaggagacggcggcggttgtgctcaaggaggtggaggtggagatggagatggtcggcggctctgaagaagcctcggcggcgccgctcctcctcgcgcacccgtgctcgctgctgcagctcctgctccgcgcctgcgccggctgcctggtgcgcctgctgcacggccactgcagcgacggcgccaacgacgacccaaaagctgctgccgacgacgacgacgctgcgcctgaagctgctgctgctgcggcggcggcggcgggcgatggcggcgacaaggcagccacctacttgtacatgcaggaggtgtgggcagtgaggaggaggccgacgacgcccggccgtccgagagaaggttccggtggcaatggagggaaccaccactag

SEQ ID NO: 34 - DNA, 516 bp, Zea mays
atggatgagcacggggaaaaggaagagaacaagtctcaagattcggctttggcggcggagcagcgcgaggagacggcggcggcggagggagaggacacgtctgaggaatccacggaccagcgcgaggacgggcacgggtataaagcggacgaatcggcgggcctgctgcccgaggacgacgtgggctctggagaagcctcggcggcgccacacttcgggcacccgtgctcgttgttgcgcgcctgcgccggattcctgggcctgcacggctgcggcggcgatcagaagccggcggcggctgccgttgctgcatctgcagctgaagccgccacggcggcggcggcgagctcgtcccaggatgaagaagacggcggcgtcgacatggcgaaggctaacggtttctacatgcatgaggtgatcacccgcgtatgggcggtcgcggtgaggaggccgcggccgcggccgccggatcacgcgagagaagggagcggtggcaatggaggcgtacaccactag

SEQ ID NO: 35 - DNA, 600 bp, Zea mays
atggcgttgtcgccgtctgcgcaagcgacaagcccgctcgcgcgccagctgctgcgccacgtgtcgtccggcctggtcgccgccgccttccaccgcccagctccagccatcagtctgccaagcgcgccgcggggagcggacgacgtggccctggcggcgtctcaccaccagcggctctggcccgctccgtcaccgaaagggcgccccggagccccgaggcaggggagcggcgggcaagtccaccacgcagctccggcgatggcgacgatagcaagcgcggttatgctgcccacgaacgccgtgacggcggcggcggtgaccacgcacgtggtgacgcgaggacccgcccggcctggtctcccggcacaaggcggcggcggcaaaacgcatggcgcaccggcggcggcgaaagcgcgcgcggccttgcgaggacctgctccgccggcccgccctaaggaaggcagcggcggaaaggttcatgtcgtgtccccggcggcgatgtcgagcgcaagtgtgcttatgcgagggcccgcgccgcctggtcacccggcagaaggcgccggcggccgtggtggaagcatccacgctatctcttcttga

SEQ ID NO: 36 - DNA, 970 bp, Arabidopsis thaliana
  misc_feature (36)..(37): N = ANY NUCLEOTIDE
tttgtgtatgaattttactatggacccaccaacccnnaatccgcnntgtggttatgttttgttgttctttttttttctccccccccctccgccccggcaggtacgatagtcgcaggccgcgccccgctccccccgcccggtcttgttgtcttattgagggtgttggcgttgtgttagtaatttacgctatttgtttagtagattttatgccaccttgcgcaagggggtacctacccttcccaccgctggttatgttgtggtggtggtccttcccttaccggtttgtcctcttttcccttcctccccgttggtgtttgccttgagctcgtgaggcatctgtggtctcggttgggcacgctgtatgagtgtttatttggtgtggagtgtggggccgggcggtgtcgcgttgttggcgtggtgtgtaggtgtgtcgtgggtgcaccgtcgtgcctccccctcctcttctgactttcttctgacttgtatattcttggtgttgttggtatgcttgacccgtctctcctctcagggcggcacccccgggtccccgtcttgtatgtctgtgcttgcgtgattctttgactgccgcggtggtttctctctctcttcctacgtgggccgcttggtaccgtgcccctgttcccgggcgcccgcgcccacagtcgctgtgcttcacttcgtgtgaccttgttcatcgtctttgctggtcccctcgccctccgttgtccttggcgttgaccgcccccccctgcccgcgcgctgcttcctgtctcgacgtcgatatcgggctcatgtggctccttgctgtccgcttctgcttttgttttctgccgcgctcgctgcgcttttttgcccgccggtattgcatgcaggcgttgatattctagttagcctagcccccggcacattattccgctcatcacgatgcccgactatcctttgcgcgcgctgtgttgtccgcgccgatacn

SEQ ID NO: 37 - DNA, 433 bp, Brassica napus
actcaccttacaagaacagcttcaattctctcactaaacttaatcatccgactagtaaaagtcttaactcagatctcatcccaatggaaaaaattgagagacaaaccgaagaagcatcttatctagggcttccttttaatttcctcaaccaaactcttaaagctatcttaaggtgtcttggaatacttcgtcatgatcctcccacggttacgaaaacgtcgtcagattctgcacccttaaaccagccggaggaggaagaagatgtggtcatggaagacaacgtcgttgtggcgactatgggcagtaaaaacggcatcataataacgagtaggggatcaaaggtgaaagcaaagaaaaaggacaaggaaaaagttagctcaggacgaccggggcaacatcactagcagtttcactacattactgctccttcttt

SEQ ID NO: 38 - DNA, 598 bp, Gossypium hirsutum
catttactgagtcgacagcgagtgtgaaacatcctctgaaagcgaaactacaaaggaatgttcatcatcatcatccccttcttcttcttcttcttcttcttttagattctaattccaaaaaaacccaaaacaacccgattttttttcacaggtaaaatcttaaactatgggtggggtgacgtcatcaatggctgcaaagtttgcgtttttcccacccaacccaccttcatacaagctggttaaagacgaagcaaccgggatttcagttttagaccctttccctcaccgtgaaaacgtcgacgtattgcgtatcccgactcgccgtggtaacgaaatcgttgccgtttacgttaggaaccccatggctacctccactctgctttattcacatgggaacgccgctgatattggtcaaatgtatgagcttttcatcgaactcagcatccatttaagaatcaatctcatggggtatgattactctggctatggacaatcatcgggaaagcctagtgagcataatacgtatgccgacatcgaagctgcatacaagtgtcttgaagagagctatggtgccaagcaagaaaacatcatcctttac

SEQ ID NO: 39 - DNA, 507 bp, Oryza sativa
atggcgtcgccgacctcgccgtcgtcgttcctcccggcgcacctcctgcggccccacgccgcgtcgctcgccggcgcaaacgtgttggtgagggacgctccgcctgagacaggcgggggcccgcaccacaacgccgtcctccgacagccgccggtgatgttggcggcggcggcgggcactccggagcaaggcagtggcccacactacaacgcggtcacgcagtggaagcccaggggaggagatcaactccgactgcccgcgtcgccgccggtgatcttggcggcggcgagcactccggagcaaggcaacggccccaagaccaacgccgtcctcaggcggccaacgccgcccggtggcgccggacctagagaaggcagcggcggacgcggcggagtgatccacgccgtcgccgactccgcgccgcagaggccaggcgcgccggcggagggcgccggcggcaatggtggagctgtccatgtcgcccctgctgctgctgcctcttcttga

SEQ ID NO: 40 - DNA, 414 bp, Oryza sativa
atggcatcagccttggcgccgttcctgatccccgcacacctcctgcagccccacgccgcgtcggcgtcttccggcctgcagctcgccggcgcaaacgtgttgctgagggacgacgctccgcccgagggaggccgaggcccgcaccacaatgccgtcctcctcccacagccgccggtgatgttggcggcggcggcgggcactccggagcaaggcaacggccccaagatcaacgccgtcctcaggcggcccacgccgcccggtggcgccggacctagagaaggcagcggcggacgcggcggagtgatccacgccatcgccgactccgcgccgcagaggccaggcgcgccggcggagggcgccggcggcaatggtggagatgtccatgtcgctcctgctgctgctacctcttcttga

SEQ ID NO: 41 - DNA, 549 bp, Oryza sativa
atggcatcagccttggcatcgccgacctcgccgtcgttcctcccggctcacctcctgcggccccacgccgcgtcgtcgtcttccggcctgcagctcgccggcgcaaaagtgttggtgagggacgctccgcccgagacaggcggaggcccgcaccacaatgccatcctcctccgacagccgccggtgatgctggcggcggcggcgggcattccggagcaaggcagtggcccacaccacaacgcggtcccgcagtggaagcccaggggaggaggagaactccgactgcccgcgtcgccgccggtgatcttggcggcggcgggcaccactccggagagaggcaacggctccaagaccaacgccgtcctcaggcggcccacgccgcccggtggcgccggacctagagaaggccgcggcggacgcggcggcgtgatccacgccgtcgccgactccgcgccgcagaggccaggctcgccggcggagggcgccggcggcaatggtggagctgtccatgccgctcctgctgctgctgctgctgccgcctcttcttga

SEQ ID NO: 42 - DNA, 519 bp, Oryza sativa
atggcctccttcatggcactggccggcgcgtgccgccgccggccgcagctcctccggccatcggcggctcgccgcctcagcttcgtcttcctcagtcgtccatttcctcctgctcctgcacccttgttgcagctgatccagcggcagcgccatcgttcttcttcttcttccagcatctcggagaacggtgcggcagcgccggtgaccgagaatctgcaggcggcggcggcgaggcggcggaggcaatcggccggcggccgcccggcagctccgaggggaggccgcggcggcggcggcgtgagaccgacgccgcccggtaacccgagagaagcgcagaagggcggcggggtgatccacgcggtggcgccgccaccggcagcgccgacgagcagcaagcctaagcctgagccgccgggctacccgagggaaggcggcggcggcaatggcggagtcgtcgacgatgtttctccttcttctaccgatacctcgacgtcttcttcttcttcctcctcctcttga

SEQ ID NO: 43 - DNA, 543 bp, Oryza sativa
atgaaggagaatacattccaggaaggaacagccatggcgtcgtcctcctcatcatcgtcctttctcgctcatccatcgtcgctgctgcgtcaggtcgtccatggcttcgccggctacctcgccggcctctgccgttctctccagaatctgaaaccatctgcagctccgaaacaagacgctgacgacgaatttgccgtcaataacactgctgcgtcaagctccgtaagtaaaacatacaactgtaccaattaatttcaataaatttcatgtaattggttcgttcaaagaacaataaatttcatgtaattgcatgatctagccatctaggtgctcaattgagcgagcccagtgattgatcagcaagtttggttgctttttttttggtcttctgagctgatgattaattttggtgtgtccttgtgagagcaggaagaagttgagaatgttcagatgagaacaagagcaatgccgcgttctgaaaggccggttttaagagaagggaacgggggaaaaggcggagcacaccacaacgctggcctttag

SEQ ID NO: 44 - DNA, 705 bp, Triticum aestivum
agttagctcgaaagaggagtgcagggcatcatctactctcgctctcgaccgcgatcaccgagacggtgaacagtgaactgcctcgacggccacgcgcagcaatggcgtccaccgctccacccgctttcctcccccagctggttcagctggtacagtctgtgtcggtcctgcccgaccagctgacggagagatcgccgccacctcagagggaaggccgcagcggggcgatcgcccccacgctgcagccatctagcgccccggtggaaggcaccggcggccaggtcatggtccttaacgacgcttcttccctggccccgcagctgaggcggacgtctcccggggaaggcacaagcgggaggatccatcgtcagcttgccggacccaagcggcctgacccgagagaaggccgcggtggcggcggcggcgcgatccatgctttttcctcttgagttctactactacccataagccagatagttcgcagatggagggtctactgtcggatgtctagtgcggtaacacagagttgcaaagttaaaagttgttgaagtgaaataaataaattagaaaagttatgatttgttaaattagttttataaaagggggttaaggcaaaaaaagagggccacaattggtaaaaaaaaaaaaatggaataatggaaaaatgtggtaaaattaaataaaatcatttggggtttaatatcc

SEQ ID NO: 45 - protein, 92 aa, Arabidopsis thaliana
MEKSDRRSEESHLWIPLQCLDQTLRAILKCLGLFHQDSPTTSSPGTSKQPKEEKEDVTMEKEEVVVTSRATKVKAKQRGKEKVSSGRPGQHN

SEQ ID NO: 46 - DNA, 499 bp, Arabidopsis thaliana
actcacatataaaaaacagcttcactcctctcaccaaaactaatcagattaataaaagttttcctctgtcttatcagatctcaatggagaaatcagatagacgaagcgaagaaagtcacctatggattcctcttcagtgcctcgaccaaaccctcagagctatcttgaaatgccttggtctttttcatcaagattctccgacaacgtcctctcccggaacttcgaaacagccgaaggaggaaaaagaagacgttaccatggaaaaggaggaggtcgttgtgacgagtagagccacaaaggtcaaggcaaagcaaagggggaaggagaaagttagctcaggccgtcctggccaacataattaggcactttaagttacattgtttagtctaattatttgcagtcgaaatgtgttaatttaatatcactgttttacttttttattatatcaacaatctacagacaaacaaaatttcattaagttcttgttcactatacgagt

SEQ ID NO: 47 - protein, 23 aa, Arabidopsis thaliana
ATKVKAKQRGKEKVSSGRPGQHN

SEQ ID NO: 48 - protein, 168 aa, Oryza sativa
MASPTSPSSFLPAHLLRPHAASLAGANVLVRDAPPETGGGPHHNAVLRQPPVMLAAAAGTPEQGSGPHYNAVTQWKPRGGDQLRLPASPPVILAAASTPEQGNGPKTNAVLRRPTPPGGAGPREGSGGRGGVIHAVADSAPQRPGAPAEGAGGNGGAVHVAPAAAASS

SEQ ID NO: 49 - protein, 137 aa, Oryza sativa
MASALAPFLIPAHLLQPHAASASSGLQLAGANVLLRDDAPPEGGRGPHHNAVLLPQPPVMLAAAAGTPEQGNGPKINAVLRRPTPPGGAGPREGSGGRGGVIHAIADSAPQRPGAPAEGAGGNGGDVHVAPAAATSS

SEQ ID NO: 50 - protein, 182 aa, Oryza sativa
MASALASPTSPSFLPAHLLRPHAASSSSGLQLAGAKVLVRDAPPETGGGPHHNAILLRQPPVMLAAAAGIPEQGSGPHHNAVPQWKPRGGGELRLPASPPVILAAAGTTPERGNGSKTNAVLRRPTPPGGAGPREGRGGRGGVIHAVADSAPQRPGSPAEGAGGNGGAVHAAPAAAAAAASS

SEQ ID NO: 51 - protein, 172 aa, Oryza sativa
MASFMALAGACRRRPQLLRPSAARRLSFVFLSRPFPPAPAPLLQLIQRQRHRSSSSSSISENGAAAPVTENLQAAAARRRRQSAGGRPAAPRGGRGGGGVRPTPPGNPREAQKGGGVIHAVAPPPAAPTSSKPKPEPPGYPREGGGGNGGVVDDVSPSSTDTSTSSSSSSSS

SEQ ID NO: 52 - protein, 115 aa, Triticum aestivum
MASTAPPAFLPQLVQLVQSVSVLPDQLTERSPPPQREGRSGAIAPTLQPSSAPVEGTGGQVMVLNDASSLAPQLRRTSPGEGTSGRIHRQLAGPKRPDPREGRGGGGGAIHAFSS

SEQ ID NO: 53 - DNA, 348 bp, Artificial Sequence
  Synthetic sequence gmPRO1PEP2
atggaagggtcttcaccatccattgaagaagagagaacagccactttctatgtgtaccatccttgctattttcttcaacaagcactcagggctctcttgaagtgtgtaggtattgatgagtctgaaaacacaatgtgttcacaggccaataaacaagagaaaagctcactgccacaaactccttctgcagatgatcctattacaaactctccaacccacaaaagctccccagatgctgcagatccaccttccacaactaatcaaaccattatcattgcaagttcaatggcaaggcgtggaaatcgagggtctagaattagccatgggtcaggccctcagcataattag

SEQ ID NO: 54 - DNA, 348 bp, Artificial Sequence
  Synthetic sequence gmPRO1atPEP1
atggaagggtcttcaccatccattgaagaagagagaacagccactttctatgtgtaccatccttgctattttcttcaacaagcactcagggctctcttgaagtgtgtaggtattgatgagtctgaaaacacaatgtgttcacaggccaataaacaagagaaaagctcactgccacaaactccttctgcagatgatcctattacaaactctccaacccacaaaagctccccagatgctgcagatccaccttccacaactaatcaaaccattatcattgccacaaaggtcaaggcaaagcaaagggggaaggagaaagttagctcaggccgtcctggccaacataattag

SEQ ID NO: 55 - DNA, 357 bp, Artificial Sequence
  Synthetic sequence gmPRO2PEP1
atggaagggtcttcagcatcatcgcatgaagaagagagaacagccactttctatgtgtaccatccttgctattttcttcaacaagcattcagggctctcttgaggtgtctaggtattgagtctgaagccacaatgtgttcaaaggcagaagaagagaaaagctcactgtcacaaactactgctgcagatgatcttattacaaactctccaagctgcaacatatcccacaaaaactcccaagatgctgcagatccaccatccacaactaatcaaaccattatcattgcaagtttaatggcaacgcgtggcagtcgagggtctaaaattagcgatgggtcaggccctcagcataattag

SEQ ID NO: 56 - DNA, 357 bp, Artificial Sequence
  Synthetic sequence gmPRO2atPEP1
atggaagggtcttcagcatcatcgcatgaagaagagagaacagccactttctatgtgtaccatccttgctattttcttcaacaagcattcagggctctcttgaggtgtctaggtattgagtctgaagccacaatgtgttcaaaggcagaagaagagaaaagctcactgtcacaaactactgctgcagatgatcttattacaaactctccaagctgcaacatatcccacaaaaactcccaagatgctgcagatccaccatccacaactaatcaaaccattatcattgccacaaaggtcaaggcaaagcaaagggggaaggagaaagttagctcaggccgtcctggccaacataattag

SEQ ID NO: 57 - protein, 115 aa, Artificial Sequence
  Synthetic peptide gmPRO1PEP2
MEGSSPSIEEERTATFYVYHPCYFLQQALRALLKCVGIDESENTMCSQANKQEKSSLPQTPSADDPITNSPTHKSSPDAADPPSTTNQTIIIASSMARRGNRGSRISHGSGPQHN

SEQ ID NO: 58 - protein, 115 aa, Artificial Sequence
  Synthetic polypeptide gmPRO1atPEP1
MEGSSPSIEEERTATFYVYHPCYFLQQALRALLKCVGIDESENTMCSQANKQEKSSLPQTPSADDPITNSPTHKSSPDAADPPSTTNQTIIIATKVKAKQRGKEKVSSGRPGQHN

SEQ ID NO: 59 - protein, 118 aa, Artificial Sequence
  Synthetic polypeptide gmPRO2PEP1
MEGSSASSHEEERTATFYVYHPCYFLQQAFRALLRCLGIESEATMCSKAEEEKSSLSQTTAADDLITNSPSCNISHKNSQDAADPPSTTNQTIIIASLMATRGSRGSKISDGSGPQHN

SEQ ID NO: 60 - protein, 118 aa, Artificial Sequence
  Synthetic polypeptide gmPRO2atPEP1
MEGSSASSHEEERTATFYVYHPCYFLQQAFRALLRCLGIESEATMCSKAEEEKSSLSQTTAADDLITNSPSCNISHKNSQDAADPPSTTNQTIIIATKVKAKQRGKEKVSSGRPGQHN

(19) United States
(12) Patent Application Publication
(10) Pub. No.: US 2014/0090103 A1
(43) Pub. Date: Mar. 27, 2014

(54) METHODS AND COMPOSITIONS FOR INCREASING PLANT DISEASE RESISTANCE AND YIELD

(71) Applicant: Monsanto Technology LLC, St. Louis, MO (US)

(72) Inventors: John Pitkin, Wildwood, MO (US); Steven E. Screen, St. Louis, MO (US); Zhidong Xie, Maryland Heights, MO (US)

(73) Assignee: Monsanto Technology LLC, St. Louis, MO (US)

(21) Appl. No.: 14/047,632

(22) Filed: Oct. 7, 2013

Related U.S. Application Data
(62) Division of application No. 12/935,275, filed on Mar. 23, 2011, now abandoned.
(60) Provisional application No. 61/044,836, filed on Apr. 14, 2008.

(51) Int. Cl. C12N 15/82; A23L 1/20; A23L 1/10; A23K 1/00; C07K 14/415; A01N 37/46

(52) U.S. Cl. CPC C12N 15/8271; C07K 14/415; A01N 37/46; A23L 1/10; A23K 1/007; A23L 1/1041; A23L 1/2006
USPC 800/278; 536/23.6; 435/320.1; 530/378; 504/335; 800/298; 800/320.1; 800/312; 800/314; 800/306; 800/320.2; 800/320.3; 800/322; 435/419; 554/9; 530/376; 530/372; 530/370; 426/443; 426/18; 426/623; 426/622; 426/618; 426/634

(57) ABSTRACT

The present invention discloses novel plant homologs of the Arabidopsis peptide atPEP1. atPEP peptides in Arabidopsis are involved in the amplification of defense pathways involved in innate immunity against microbial pathogens. Homologs to atPEP1 identified in soy, corn, rice, wheat, and canola sequence databases are potential sources for transgenes to enhance crop yield through resistance to biotic and/or abiotic stresses. Chimeric genes comprising sequences from mature and precursor plant defense response polypeptides from a given species, and from different species, as well as the encoded polypeptide sequences, are also described.

### CROSS REFERENCE TO RELATED APPLICATIONS

This application claims the priority of U.S. Provisional Application Ser. No. 61/044,836 filed Apr. 14, 2008, the entire disclosure of which is incorporated herein by reference.

### INCORPORATION-BY-REFERENCE OF SEQUENCE LISTING IN COMPUTER READABLE FORM

The Sequence Listing, which is a part of the present disclosure, includes a computer readable form 42 KB file (as measured in Microsoft Windows®) created on Apr. 10, 2009 and entitled “MONS201WOsequencelisting.txt” comprising nucleotide sequences of the present invention. The subject matter of the Sequence Listing is incorporated herein by reference in its entirety.

### BACKGROUND OF THE INVENTION

1. Field of the Invention

The present invention relates generally to enhancing plant responses to biotic and abiotic stresses, and in particular to identifying elicitors of defense signaling in plants.

2. Description of Related Art

Plants are subject to multiple potential stresses, diseases, and pests, including, among others, abiotic stresses such as temperature stress, moisture stress, and nutrient stress, as well as biotic stresses caused by various microbial pathogens, parasites, attack by insects and other pests, and herbivory. Biochemical and molecular responses of plants to such stresses have been studied in order to enhance plant growth and crop yield in the face of such factors which would otherwise impair the desired growth and yield.

### SUMMARY OF THE INVENTION

In one aspect, the invention provides an isolated polynucleotide comprising a sequence selected from the group consisting of: a) a polynucleotide sequence at least 75% identical to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:53, SEQ ID NO:54, SEQ ID NO:55, or SEQ ID NO:56; b) a polynucleotide encoding a polypeptide at least 85% identical to SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51 or SEQ ID NO:52; and c) a polynucleotide that hybridizes to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44, or a complement thereof, under stringent wash conditions of 0.2×SSC at 65° for 10 minutes. In one embodiment, the isolated polynucleotide sequence comprises SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44 SEQ ID NO:53, SEQ ID NO:54, SEQ ID NO:55, or SEQ ID NO:56. In another embodiment, the isolated polynucleotide sequence of claim 1, wherein the polynucleotide encodes a polypeptide selected from the group consisting of SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51 and SEQ ID NO:52

Another embodiment of the invention provides a construct comprising an isolated polynucleotide as described herein, operably linked to a heterologous promoter functional in plants. In certain embodiments the promoter is a stress induced promoter, and in particular embodiments is selected from the group consisting of a biotic stress inducible promoter or an abiotic stress inducible promoter, including a pathogen inducible promoter, an osmotic stress inducible promoter, or a temperature inducible promoter. In certain embodiments, the promoter is tissue specific or developmentally specific. In other embodiments, the promoter is constitutive.

Another aspect of the invention provides an isolated polypeptide sequence comprising an amino acid sequence polypeptide at least 85% identical to SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51 or SEQ ID NO:52.

Yet another aspect of the invention provides a composition formulated for application to a plant or a part thereof comprising the polypeptide as described above. In certain embodiments the composition is formulated as a spray, a powder, a granule, or a seed treatment.

An additional aspect of the invention provides a method for improving the health of a plant, comprising providing to the plant a polypeptide as described herein in an amount that improves the health of the plant as compared to a plant of the same genotype not provided with the polypeptide. In certain embodiments, providing the polypeptide comprises contacting the plant with the composition as described, is formulated as a spray, a powder, a granule, or a seed treatment. In other embodiments, providing the polypeptide comprises expressing in the plant a nucleic acid encoding the polypeptide as described herein.

A transgenic plant or a part thereof transformed with the polynucleotide as described herein is another aspect of the invention. In certain embodiments, the plant is selected from the group consisting of corn, soybean, cotton, canola, rice, wheat, and sunflower. In other embodiments, the part of the plant, wherein the part comprises the polynucleotide as described is selected from the group consisting of an embryo, pollen, a cell, a root, a fruit or a meristem. Another embodiment of the invention provides seed of the plant, wherein the seed comprises comprising a sequence selected from the group consisting of: a) a polynucleotide sequence at least 75% identical to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:53, SEQ ID NO:54, SEQ ID NO:55, or SEQ ID NO:56; b) a polynucleotide encoding a polypeptide at least 85% identical to SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51 or SEQ ID NO:52; and c) a polynucleotide that hybridizes to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:53, SEQ ID NO:54, SEQ ID NO:55, or SEQ ID NO:56, or a complement thereof, under stringent wash conditions of 0.2×SSC at 65° for 10 minutes.

A method of producing a plant commodity product, comprising producing the commodity product from a plant comprising a polynucleotide as described herein thereof is a further aspect of the invention. In certain embodiments, the commodity product is selected from the group consisting of grain, meal, protein, flour, oil, or silage. In particular embodiments, the commodity product produced by the method comprises a polynucleotide as described herein.

### DESCRIPTION OF SEQUENCE LISTINGS

SEQ ID NO:1 G. max gmPROPEP1 polypeptide sequence

SEQ ID NO:2 G. max gmPROPEP2 polypeptide sequence

SEQ ID NO:3 G. max gmPROPEP3 polypeptide sequence

SEQ ID NO:4 Z. mays zmPROPEP2 polypeptide sequence

SEQ ID NO:5 Z. mays zmPROPEP3 polypeptide sequence

SEQ ID NO:6 Z. mays zmPROPEP4 polypeptide sequence

SEQ ID NO:7 G. max gmPEP1 predicted mature polypeptide sequence

SEQ ID NO:8 G. max gmPEP2 predicted mature polypeptide sequence

SEQ ID NO:9 G. max gmPEP3 predicted polypeptide sequence fragment

SEQ ID NO:10 G. max gmPEP3 predicted mature polypeptide sequence

SEQ ID NO:11 Z. mays zmPEP1 predicted mature peptide sequence

SEQ ID NO:12 Z. mays zmPEP2 predicted mature polypeptide sequence

SEQ ID NO:13 Z. mays zmPEP4 predicted mature polypeptide sequence

SEQ ID NO:14 A. thaliana atPEP8 predicted polypeptide sequence fragment

SEQ ID NO:15 A. thaliana atPEP8 predicted mature polypeptide sequence

SEQ ID NO:16 B. napus bnPEP2 predicted mature polypeptide sequence

SEQ ID NO:17 G. hirsutum ghPEP1 predicted polypeptide sequence fragment

SEQ ID NO:18 G. hirsutum ghPEP1 predicted mature polypeptide sequence

SEQ ID NO:19 O. sativa osPEP3 predicted polypeptide sequence fragment

SEQ ID NO:20 O. sativa osPEP3 predicted mature polypeptide sequence

SEQ ID NO:21 O. sativa osPEP4 predicted polypeptide sequence fragment

SEQ ID NO:22 O. sativa osPEP4 predicted mature polypeptide sequence

SEQ ID NO:23 O. sativa osPEP5 predicted polypeptide sequence fragment

SEQ ID NO:24 O. sativa osPEP5 predicted mature polypeptide sequence

SEQ ID NO:25 O. sativa osPEP6 predicted polypeptide sequence fragment

SEQ ID NO:26 O. sativa osPEP6 predicted mature polypeptide sequence

SEQ ID NO:27 O. sativa osPEP7 predicted polypeptide sequence fragment

SEQ ID NO:28 O. sativa osPEP7 predicted mature polypeptide sequence

SEQ ID NO:29 T. aestivum taPEP3 predicted polypeptide sequence fragment

SEQ ID NO:30 T. aestivum taPEP3 predicted mature polypeptide sequence

SEQ ID NO:31 G. max gmPROPEP1 ORF

SEQ ID NO:32 G. max gmPROPEP2 ORF

SEQ ID NO:33 Z. mays zmPROPEP3 ORF

SEQ ID NO:34 Z. mays zmPROPEP2 ORF

SEQ ID NO:35 Z. mays zmPROPEP4 ORF

SEQ ID NO:36 A. thaliana MRT3702_{—}140538C cDNA contig encoding AtPEP8

SEQ ID NO:37 B. napus MRT3708_{—}29412C cDNA contig encoding bnPEP2

SEQ ID NO:38 G. hirsutum MRT3635_{—}34589C cDNA contig encoding ghPEP1

SEQ ID NO:39 O. sativa Os08g07630 osPEP3 coding sequence

SEQ ID NO:40 O. sativa Os08g07640 osPEP4 coding sequence

SEQ ID NO:41 O. sativa Os08g07660 osPEP5 coding sequence

SEQ ID NO:42 O. sativa Os08g07670 osPEP6 coding sequence

SEQ ID NO:43 O. sativa Os08g07690 osPEP7 coding sequence

SEQ ID NO:44 T. aestivum MRT4565_{—}89997C taPEP3 encoding sequence

SEQ ID NO:45 A. thaliana atPROPEP1 polypeptide sequence

SEQ ID NO:46 A. thaliana locus At5g64900 nucleotide sequence

SEQ ID NO:47 A. thaliana atPEP1 predicted mature polypeptide sequence

SEQ ID NO:48 O. sativa osPEP3 predicted precursor polypeptide sequence

SEQ ID NO:49 O. sativa osPEP4 predicted precursor polypeptide sequence

SEQ ID NO:50 O. sativa osPEP5 predicted precursor polypeptide sequence

SEQ ID NO:51 O. sativa osPEP6 predicted precursor polypeptide sequence

SEQ ID NO:52 T. aestivum taPEP3 predicted precursor polypeptide sequence

SEQ ID NO:53 Chimeric polynucleotide sequence comprising precursor region of gmPROPEP1 and mature region of gmPEP2.

SEQ ID NO:54 Chimeric polynucleotide sequence comprising precursor region of gmPROPEP1 and mature region of atPEP1.

SEQ ID NO:55 Chimeric polynucleotide sequence comprising precursor region of gmPROPEP2 and mature region of gmPEP1.

SEQ ID NO:56 Chimeric polynucleotide sequence comprising precursor region of gmPROPEP2 and mature region of atPEP1.

SEQ ID NO:57 Predicted polypeptide sequence of the residues encoded by SEQ ID NO:53.

SEQ ID NO:58 Predicted polypeptide sequence of the residues encoded by SEQ ID NO:54.

SEQ ID NO:59 Predicted polypeptide sequence of the residues encoded by SEQ ID NO:55.

SEQ ID NO:60 Predicted polypeptide sequence of the residues encoded by SEQ ID NO:56.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention.

The invention provides methods and compositions for enhancement of plant response to stresses. Novel plant homologs of the Arabidopsis peptide elicitor atPEP1 were identified. AtPEP peptides in Arabidopsis are involved in the amplification of defense pathways involved in innate immunity against pathogens and stresses. Soybean and corn homologs were also identified, as well as from rice, wheat, and canola. These sequences may be used in accordance with the invention to enhance resistance to disease and other biotic or abiotic stresses, as well as yield. Abiotic stresses may include osmotic stress (e.g. including drought stress or salt stress), cold or heat stress, oxidative stress, and nutrient deficit, among others. Biotic stresses may include, for example, infection by microbial pathogens such as those that cause fungal disease, oomycetes, viral disease, or bacterial disease; insect infestation, nematode infestation, weed infestation, and herbivory, among others.

AtPEP peptides are involved in amplifying plant responses to environmental stress(es), resulting in increased defense pathway gene expression and enhanced resistance to disease. AtPEP1 is 23 amino acid residues in length (Huffaker, 2006), while the gene encoding the AtPEP1 peptide precursor (atPROPEP1) encodes a larger 93 amino acid propeptide, which is presumably processed and secreted outside the plant cell. Transcriptional profiling analysis shows that expression of one of the corn atPROPEP1 homologs is increased in cold. This suggests a role in abiotic stress response. In specific embodiments of the invention, such polypeptide-encoding genes may be expressed as transgenes in crops to confer biotic or abiotic stress resistance. Such a transgene may be comprised within a genetic construct and be operably linked to a heterologous promoter, such as a stress inducible promoter, for appropriate expression.

Following the infection of a plant by a potential pathogen, the pathogen may successfully proliferate in the host, causing associated disease symptoms, or its growth may be halted by host plant defense. One such defense is the hypersensitive response (HR), characterized by rapid apoptotic cell death near the site of the infection that correlates with the generation of activated oxygen species, production of antimicrobial compounds, and reinforcement of host cell walls (e.g. Dixon and Lamb, 1990). Other defenses include systemic acquired resistance, which effectively protects the plant against subsequent attack by a broad range of pathogens (e.g. Ryals et al., 1995). Pathogens that elicit an HR on a given host are described as “avirulent” on that host, the host is described as “resistant,” and the plant-pathogen interaction is “incompatible.” If a pathogen proliferates and causes disease on the host, the pathogen is termed “virulent,” the host is termed “susceptible,” and the plant-pathogen interaction is “compatible.” Response of plants to abiotic stresses may also be mediated by polypeptides such as those described herein.

Both dicotyledonous and monocotyledonous plants have been found to produce such endogenous defense polypeptides. Among the crop plants contemplated for use with the present invention are corn, soybean, cotton, canola, sunflower, wheat, rice, tomato, onion, squash, cucumber, pepper, other vegetable plants, and ornamental plants. Additionally, barley, rye, potato, clover, other legume such as pea or alfalfa, sugar cane, sugar beet, tobacco, carrot, safflower, sorghum, strawberry, banana, and turfgrass are also contemplated.

Thus, transgenic crop plants and seeds with enhanced stress resistance are one aspect of the present invention, and may be produced using, for instance, using a transgene encoding the described polypeptide(s). In other embodiments, one or more of the described defense polypeptides may be synthesized in vitro, for instance using known peptide synthesis techniques, and formulated for application to a plant or plant part.

A. Nucleic Acid Compositions and Constructs

The invention provides recombinant DNA constructs for use in achieving enhanced plant response to environmental stresses, including biotic and/or abiotic stresses. Transformed host targets may express effective levels of polypeptide(s) encoded by the recombinant DNA constructs.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides or peptides.

Provided according to the invention are nucleotide sequences, the expression of which results in an RNA sequence which encodes a plant defense response polypeptide (i.e. “elicitor” polypeptide, or polypeptide precursor. In plants, the described polypeptides are typically natively produced as precursor polypeptides, that are proteolytically processed to yield bioactive polypeptide(s). Thus, for example, “gmPROPEP2” refers to such a precursor of a Glycine max gmPEP2 mature polypeptide. Multiple processed products may result from a given precursor polypeptide, which may interact with an appropriate plant receptor, whether native or heterologous, to effect a stress response.

As used herein, the term “substantially homologous” or “substantial homology”, with reference to a nucleic acid sequence, includes a nucleotide sequence that hybridizes under stringent conditions to the coding sequence of SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51 or SEQ ID NO:52, e.g. any of SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:53, SEQ ID NO:54, SEQ ID NO:55, or SEQ ID NO:56, or the complement thereof, under stringent wash conditions of 0.2×SSC at 65° for 10 minutes. Such conditions yield high selectivity, with relatively low salt and/or high temperature conditions. Other examples of such conditions include about 0.02 M to about 0.15 M NaCl at temperatures of about 50° C. to about 70° C. For example, a further high stringency condition is to wash the hybridization filter two or more times with high-stringency wash buffer (0.2×SSC or 1×SSC, 0.1% SDS, 65° C.) for 10 minutes per rinse. Other conditions in the art or can be found in Ausubel (1998). Both temperature and salt may be varied, or either the temperature or the salt concentration may be held constant while the other variable is changed.

In a further embodiment of the invention, nucleic acids are provided defined as exhibiting at least about 70% sequence identity to a nucleic acid sequence provided herein, including at least about, and specifically including at least, 75%, 80%, 85%, 88%, 90%, 93%, 95%, 97%, 98% and 99% identity with respect to a sequence provided herein and specifically including those set forth immediately herein above. In one embodiment the reference sequence is elected from SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:53, SEQ ID NO:54, SEQ ID NO:55, or SEQ ID NO:56, or a complement thereof.

In yet another embodiment of the invention, nucleic acids are provided defined as encoding a polypeptide that exhibits at least about 70% sequence identity to a polypeptide provided herein, specifically including any one or more of the polypeptides provided in the Sequence Listing and specifically including a polypeptide encoded by any nucleic acid sequence provided herein. This includes sequences encoding a polypeptide with at least about, and specifically including at least, 75%, 80%, 85%, 88%, 90%, 93%, 95%, 97%, 98% and 99% identity to any such polypeptide sequences.

As used herein, the term “ortholog” refers to a gene in two or more species that has evolved from a common ancestral nucleotide sequence, and may retain the same function in the two or more species.

As used herein, the term “sequence identity,” “sequence similarity” or “homology” is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, a “comparison window” refers to a conceptual segment of at least 6 contiguous positions, usually about 50 to about 100, more usually about 100 to about 150, in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences. Those skilled in the art should refer to the detailed methods used for sequence alignment in the Wisconsin Genetics Software Package Release 7.0, Genetics Computer Group, 575 Science Drive Madison, Wis., USA) or refer to Ausubel et al. (1998) for a detailed discussion of sequence analysis.

The present invention provides DNA sequences capable of being expressed as an RNA transcript in a cell to enhance one or more plant stress defense responses. The DNA molecule may be placed operably under the control of a promoter sequence that functions in the cell, tissue or organ of the host expressing the DNA to produce a plant defense polypeptide or polypeptide precursor. In certain embodiments, the DNA sequence may be derived from a nucleotide sequence as set forth SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44, or a complement thereof, in the sequence listing, such as SEQ ID NO:53, SEQ ID NO:54, SEQ ID NO:55, or SEQ ID NO:56. For instance, the sequence may be a chimeric polynucleotide sequence, such as SEQ ID NO:53, SEQ ID NO:54, SEQ ID NO:55, or SEQ ID NO:56, or may encode a chimeric polypeptide sequences such as SEQ ID NO:57, SEQ ID NO:58, SEQ ID NO:59, or SEQ ID NO:60.

The invention also provides a DNA sequence for expression in a cell of a plant that, upon expression of the DNA to RNA and transcription of the RNA to produce an encoded peptide or polypeptide, enhances the ability of the plant or plant cell to withstand an abiotic or biotic stress, or enhances the yield or value of the plant, or a crop or product produced from the plant.

An expression vector minimally comprises a polynucleotide sequence which encodes a polypeptide that is expressed in a host cell. Typically, an expression vector is placed under the control of certain regulatory elements including promoters, tissue specific regulatory elements, and enhancers. Such an expression vector is said to be “operably linked to” the regulatory elements. A gene sequence or fragment for plant stress control according to the invention may be operably linked to promoter, which is functional in a transgenic plant cell and therein expressed to produce mRNA in the transgenic plant cell.

As used herein “promoter” means a region of DNA sequence that is essential for the initiation of transcription of RNA from DNA. Promoters are located upstream of DNA to be transcribed and have regions that act as binding sites for RNA polymerase and have regions that work with other factors to promote RNA transcription. More specifically, basal promoters in plants comprise canonical regions associated with the initiation of transcription, such as CAAT and TATA boxes. In the present invention, preferred promoter molecules and 5′ UTR molecules allow for transcription in plant cells or tissues at a rate or level greater than in other cells and tissues of the plant. Those skilled in the art will recognize that there are a number of constitutive and tissue specific promoters that are functional in plant cells, and have been described in the literature. For example, promoters are described in Odell et al., (1985); U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. Nos. 5,322,938, 5,352,605, and 5,530,196 (35S promoter); U.S. Pat. No. 6,429,357 (rice actin 2 promoter as well as a rice actin 2 intron); U.S. Pat. No. 6,294,714 (light inducible promoters); U.S. Pat. No. 6,140,078 (salt inducible promoters); U.S. Pat. No. 6,252,138 (pathogen inducible promoters); U.S. Pat. No. 6,175,060 (phosphorus deficiency inducible promoters); all of which are incorporated herein by reference. In certain embodiments, the promoter utilized for expression of the plant defense response polypeptide is a stress-inducible promoter.

Polypeptides of interest for improving plant tolerance to cold or freezing temperatures include, among others, polypeptides involved in biosynthesis of trehalose or raffinose, polypeptides encoded by cold induced genes, fatty acyl desaturases and other polypeptides involved in glycerolipid or membrane lipid biosynthesis, which find use in modification of membrane fatty acid composition, alternative oxidase, calcium-dependent protein kinases, LEA proteins and uncoupling protein. Thus, the promoter from such a gene may be useful in the present invention. Polypeptides of interest for improving plant tolerance to heat include, among others, polypeptides involved in biosynthesis of trehalose, polypeptides involved in glycerolipid biosynthesis or membrane lipid metabolism (for altering membrane fatty acid composition), heat shock proteins and mitochondrial NDK. Thus, the promoter from such a gene may be useful in the present invention. Polypeptides of interest for improving plant tolerance to extreme osmotic conditions include, among others, polypeptides involved in proline biosynthesis. Further, polypeptides of interest for improving plant tolerance to drought conditions include, among others, aquaporins, polypeptides involved in biosynthesis of trehalose or wax, LEA proteins and invertase. Thus, the promoter from such genes may be useful in the present invention. Polypeptides of interest for improving pathogen or pest tolerance to effects of plant pests or pathogens include, among others, proteases, polypeptides involved in anthocyanin biosynthesis, polypeptides involved in cell wall metabolism, including cellulases, glucosidases, pectin methylesterase, pectinase, polygalacturonase, chitinase, chitosanase, and cellulose synthase, and polypeptides involved in biosynthesis of secondary compounds such as terpenoids or indole for production of bioactive metabolites to provide defense against herbivorous insects. Thus, the promoters from such genes may be useful in the present invention.

The nucleic acid molecules or fragments of the nucleic acid molecules or other nucleic acid molecules in the sequence listing are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are well known in the art, and are described, for instance, by Sambrook, et al. (1989), and by Haymes et al. (1985).

Departures from complete complementarity are therefore permissible, as long as such departures do not completely preclude the capacity of the molecules to form a double-stranded structure. Thus, in order for a nucleic acid molecule or a fragment of the nucleic acid molecule to serve as a primer or probe it needs only be sufficiently complementary in sequence to be able to form a stable double-stranded structure under the particular solvent and salt concentrations employed.

Nucleic acids and peptides of the present invention may also be synthesized, either completely or in part, especially where it is desirable to provide sequences comprising a given plant's preferred codon frequencies, by methods known in the art. Thus, all or a portion of the nucleic acids of the present invention may be synthesized using codons preferred by a selected host. Species-preferred codons may be determined, for example, from the codons used most frequently in the proteins expressed in a particular host species. Other modifications of the nucleotide sequences may result in mutants having slightly altered activity.

Another aspect of the invention relates to a DNA construct comprising a nucleotide sequence provided herein that encodes a plant defense polypeptide, such as those described herein above. The present invention further provides plant defense polypeptides comprising a polypeptide sequence described herein and/or encoded by a nucleic acid provided by the invention. In one embodiment, the polypeptide sequence may comprise a sequence elected from SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51, SEQ ID NO:52, SEQ ID NO:57, SEQ ID NO:58, SEQ ID NO:59, or SEQ ID NO:60. In certain embodiments, the polypeptide may be defined as exhibiting at least 70%, 75%, 80%, 85%, 88%, 90%, 93%, 95%, 97%, 98%, or 99% or greater percent sequence identity to any one of more of such sequences. The invention also provides compositions formulated for application to a plant containing any such polypeptide including all derivable combination(s) thereof.

The invention further relates to methods for improving the health of a plant, by providing such plant defense response polypeptides to a plant. In certain embodiments “providing” comprises transforming cells of a plant with polynucleotides that function to produce such plant defense response polypeptide(s). Plant health may be improved as compared to a plant of otherwise the same genotype but not provided with the polypeptide.

In other aspects of the invention, a transgenic plant or part thereof, including a seed, that comprises an isolated a polynucleotide sequence provided herein. In certain embodiments, the plant is a crop plant, such as a corn, cotton, soybean, wheat, rice, or canola plant. In other embodiments, the plant part is a seed of such a crop plant.

Still another aspect of the invention relates to plant commodity products and methods for producing plant commodity products, produced from a plant or plant part as described herein. For instance, the commodity product may be, among others, grain, meal, forage, protein, isolated protein, flour, oil, or silage, wherein the crop from which it is produced comprises a polynucleotide sequence provided by the invention operably linked to a heterologous promoter.

Commodity products containing one or more of the sequences of the present invention, and produced from a recombinant plant or seed containing one or more of the nucleotide sequences of the present invention are specifically contemplated as embodiments of the present invention. A commodity product containing one or more of the sequences of the present invention is intended to include, but not be limited to, meals, oils, crushed or whole grains or seeds of a plant, or any food product comprising any meal, oil, or crushed or whole grain of a recombinant plant or seed containing one or more of the sequences of the present invention. The detection of one or more of the sequences of the present invention in one or more commodity or commodity products contemplated herein is defacto evidence that the commodity or commodity product is composed of a transgenic plant designed to express one or more of the plant defense response polypeptide sequences of the present invention for the purpose of controlling plant stress, including enhancing resistance to a biotic or abiotic stress.

B. Recombinant Vectors and Host Cell Transformation

A recombinant DNA vector may, for example, be a linear or a closed circular plasmid. The vector system may be a single vector or plasmid or two or more vectors or plasmids that together contain the total DNA to be introduced into the genome of a bacterial host, for use in subsequent plant cell transformation. For instance, nucleic acid molecules provided herein and complements or fragments thereof, can, for example, be suitably inserted into a vector under the control of a suitable promoter that functions in a host plant to drive expression of a linked coding sequence or other DNA sequence. Many vectors are available for this purpose, and selection of the appropriate vector will depend mainly on the size of the nucleic acid to be inserted into the vector and the particular host cell to be transformed with the vector. Each vector may contain various components depending on its function (amplification of DNA or expression of DNA) and the particular host cell with which it is compatible. The vector components for bacterial or plant transformation generally include, but are not limited to, one or more of the following: a signal sequence, an origin of replication, one or more selectable marker genes, and a promoter, such as an inducible promoter, allowing the expression of exogenous DNA.

Expression and cloning vectors generally contain a selection gene, also referred to as a selectable marker. This gene encodes a protein necessary for the survival or growth of transformed host cells grown in a selective culture medium. Typical selection genes encode proteins that (a) confer resistance to antibiotics or other toxins, e.g., ampicillin, neomycin, methotrexate, spectinomycin, or tetracycline, (b) complement auxotrophic deficiencies, or (c) supply critical nutrients not available from complex media, e.g., the gene encoding D-alanine racemase for Bacilli. Those cells that are successfully transformed with a heterologous protein or fragment thereof produce a protein conferring drug resistance and thus survive the selection regimen.

An expression vector for producing an mRNA encoding a plant defense response polypeptide or a precursor of a plant defense response polypeptide can also contain an inducible promoter that is recognized by a host plant cell and is operably linked to the nucleic acid. Inducible promoters suitable for use with the presently described polypeptide-encoding sequences include, for instance, those described in U.S. Pat. No. 6,252,138. However, other known promoters are suitable.

The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream (5′ noncoding sequences), within, or downstream (3′ non-translated sequences) of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, and polyadenylation recognition sequences and the like.

Construction of suitable vectors containing one or more of the above-listed components employs standard recombinant DNA techniques. Isolated plasmids or DNA fragments are cleaved, tailored, and re-ligated in the form desired to generate the plasmids required. Examples of available bacterial expression vectors include, but are not limited to, the multifunctional E. coli cloning and expression vectors such as Bluescript™ (Stratagene, La Jolla, Calif.), in which, for example, a nucleic acid, or fragment thereof may be ligated into the vector in frame with sequences for the amino-terminal Met and the subsequent 7 residues of β-galactosidase so that a hybrid protein is produced; pIN vectors (Van Heeke and Schuster, 1989); and the like.

The present invention also contemplates transformation of a nucleotide sequence of the present invention into a plant to achieve enhanced plant stress resistance. A transformation vector can be readily prepared using methods available in the art. The transformation vector comprises one or more nucleotide sequences that is/are capable of being transcribed to yield the plant defense response polypeptide or a precursor thereof, such that upon production of such a polypeptide, enhanced plant stress resistance is effected.

The transformation vector may be defined as a recombinant molecule, a disease control agent, a genetic molecule or a chimeric genetic construct. A chimeric genetic construct of the present invention may comprise, for example, nucleotide sequences encoding one or more such plant defense response polypeptides.

In one embodiment a plant transformation vector comprises an isolated and purified DNA molecule comprising a heterologous promoter operatively linked to one or more nucleotide sequences of the present invention. The DNA molecule comprising the expression vector may also contain a functional intron sequence positioned either upstream of the coding sequence or even within the coding sequence, and may also contain a five prime (5′) untranslated leader sequence (i.e., a UTR or 5′-UTR) positioned between the promoter and the point of translation initiation.

A plant transformation vector that functions as a stress defense agent, including as a disease response-enhancing agent, may contain sequences from more than one gene, thus allowing production of more than one plant defense response polypeptide for effecting enhanced stress resistance. One skilled in the art will readily appreciate that in the stress response agent of the present invention, the gene(s) encoding the plant defense polypeptide(s) can be obtained from the same plant species as is being transformed, in order to enhance the effectiveness of the control agent. In certain embodiments, the gene(s) can be derived from a different plant species.

A recombinant DNA vector or construct of the present invention may comprise a selectable marker that confers a selectable phenotype on plant cells. Selectable markers may also be used to select for plants or plant cells that contain the exogenous nucleic acids encoding polypeptides or proteins of the present invention. The marker may encode biocide resistance, antibiotic resistance (e.g., kanamycin, G418 bleomycin, hygromycin, etc.), or herbicide resistance (e.g., glyphosate, etc.). Examples of selectable markers include, but are not limited to, a neo gene which codes for kanamycin resistance and can be selected for using kanamycin, G418, etc., a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulfonylurea resistance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, and tetracycline, and the like. Examples of such selectable markers are illustrated in U.S. Pat. Nos. 5,550,318; 5,633,435; 5,780,708 and 6,118,047.

Preferred plant transformation vectors include those derived from a Ti plasmid of Agrobacterium tumefaciens (e.g. U.S. Pat. Nos. 4,536,475, 4,693,977, 4,886,937, 5,501,967 and EP 0 122 791). Other preferred plant transformation vectors include those disclosed, e.g., by Herrera-Estrella (1983); Bevan (1983), Klee (1985) and EP 0 120 516.

In general it may be preferred to introduce a functional recombinant DNA at a non-specific location in a plant genome. In special cases it may be useful to insert a recombinant DNA construct by site-specific integration. Several site-specific recombination systems exist which are known to function in plants include cre-lox as disclosed in U.S. Pat. No. 4,959,317 and FLP-FRT as disclosed in U.S. Pat. No. 5,527,695.

Suitable methods for transformation of host cells for use with the current invention are believed to include virtually any method by which DNA can be introduced into a cell (see, for example, Miki et al., 1993), such as by transformation of protoplasts (U.S. Pat. No. 5,508,184; Omirulleh et al., 1993), by desiccation/inhibition-mediated DNA uptake (Potrykus et al., 1985), by electroporation (U.S. Pat. No. 5,384,253), by agitation with silicon carbide fibers (Kaeppler et al., 1990; U.S. Pat. No. 5,302,523; and U.S. Pat. No. 5,464,765), by Agrobacterium-mediated transformation (U.S. Pat. Nos. 5,563,055; 5,591,616; 5,693,512; 5,824,877; 5,981,840; 6,384,301) and by acceleration of DNA coated particles (U.S. Pat. Nos. 5,015,580; 5,550,318; 5,538,880; 6,160,208; 6,399,861; 6,403,865; Padgette et al. 1995), etc. Through the application of techniques such as these, the cells of virtually any species may be stably transformed. In the case of multicellular species, the transgenic cells may be regenerated into transgenic organisms.

The most widely utilized method for introducing an expression vector into plants is based on the natural transformation system of Agrobacterium (for example, Horsch et al., 1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. Descriptions of Agrobacterium vector systems and methods for Agrobacterium-mediated gene transfer are provided by numerous references, including Gruber et al. 1993; Miki et al., 1993, Moloney et al., 1989, and U.S. Pat. Nos. 4,940,838 and 5,464,763. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector (Broothaerts et al., 2005).

Methods for the creation of transgenic plants and expression of heterologous nucleic acids in plants in particular are known and may be used with the nucleic acids provided herein to prepare transgenic plants that exhibit reduced susceptibility to stress. Plant transformation vectors can be prepared, for example, by inserting the dsRNA producing nucleic acids disclosed herein into plant transformation vectors and introducing these into plants. One known vector system has been derived by modifying the natural gene transfer system of Agrobacterium tumefaciens. The natural system comprises large Ti (tumor-inducing)-plasmids containing a large segment, known as T-DNA, which is transferred to transformed plants. Another segment of the Ti plasmid, the vir region, is responsible for T-DNA transfer. The T-DNA region is bordered by terminal repeats. In the modified binary vectors the tumor-inducing genes have been deleted and the functions of the vir region are utilized to transfer foreign DNA bordered by the T-DNA border sequences. The T-region may also contain a selectable marker for efficient recovery of transgenic plants and cells, and a multiple cloning site for inserting sequences for transfer such as a dsRNA encoding nucleic acid.

A transgenic plant formed using Agrobacterium transformation methods typically may contain a single simple recombinant DNA sequence inserted into one chromosome, referred to as a transgenic event. Such transgenic plants can be referred to as being heterozygous for the inserted exogenous sequence. A transgenic plant homozygous with respect to a transgene can be obtained by sexually mating (selfing) an independent segregant transgenic plant that contains a single exogenous gene sequence to itself, for example an F0 plant, to produce F1 seed. One fourth of the F1 seed produced will be homozygous with respect to the transgene. Germinating F1 seed results in plants that can be tested for heterozygosity, typically using a SNP assay or a thermal amplification assay that allows for the distinction between heterozygotes and homozygotes (i.e., a zygosity assay). Crossing a heterozygous plant with itself or another heterozygous plant results in heterozygous progeny, as well as homozygous transgenic and homozygous null progeny.

The present invention provides seeds and plants having one or more transgenic events. Combinations of events are referred to as “stacked” transgenic events. These stacked transgenic events can be events that are directed at the same target organism, or they can be directed at different target pathogens or pests.

In certain embodiments, a seed having the ability to express a plant defense response polypeptide or polypeptide precursor, also has a “stacked” transgenic event that provides herbicide tolerance. Beneficial example of herbicide tolerance genes providing resistance to herbicides include glyphosate, N-(phosphonomethyl)glycine, including the isopropylamine salt form of such herbicide, and dicamba.

In addition to direct transformation of a plant with a recombinant DNA construct, transgenic plants can be prepared by crossing a first plant having a recombinant DNA construct with a second plant lacking the construct. For example, recombinant DNA for gene suppression can be introduced into first plant line that is amenable to transformation to produce a transgenic plant that can be crossed with a second plant line to introgress the recombinant DNA for gene suppression into the second plant line.

The present invention can be, in practice, combined with other stress response including disease control traits in a plant to achieve desired traits for enhanced plant stress resistance. Combining traits that employ distinct modes-of-action can provide protected transgenic plants with superior durability over plants harboring a single control trait because of the reduced probability that resistance will develop in the field.

As used herein, the term “derived from” refers to a specified nucleotide sequence that may be obtained from a particular specified source or species, albeit not necessarily directly from that specified source or species.

As used herein, the phrase “coding sequence”, “structural nucleotide sequence” or “structural nucleic acid molecule” refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus A coding sequence can include, but is not limited to, genomic DNA, cDNA, EST and recombinant nucleotide sequences.

The term “recombinant DNA” or “recombinant nucleotide sequence” refers to DNA that contains a genetically engineered modification through manipulation via mutagenesis, restriction enzymes, and the like.

### EXAMPLES

### Example 1

### Identification of Endogenous Plant Defense Peptides

A bioinformatics tools-based approach was developed and used to identify homologs of atPEP (A. thaliana plant stress response peptide) sequences in proprietary and publicly available sequence databases. The AtPROPEP1 sequence (SEQ ID NO:45; GenBank NM12588) encoded within At5g64900 (SEQ ID NO:46), or AtPEP1 (SEQ ID NO:47; Huffaker et al., 2006; GenBank CD401281) was utilized for initial searches. One new soy and one new corn homolog were identified in proprietary databases using conventional BLAST searches. Eleven new plant homologs were identified using a position weighted motif matrix search. Table 1 describes nucleotide and amino acid sequences of newly identified endogenous plant defense peptides.

TABLE 1
Additional identified AtPEP homolog sequences.

| Sequence | Designation |
|---|---|
| SEQ ID NO: 2 | G. max PROPEP2 precursor |
| SEQ ID NO: 3 | G. max PROPEP3 precursor |
| SEQ ID NO: 4 | Zea mays PROPEP2 precursor |
| SEQ ID NO: 5 | Z. mays PROPEP3 precursor |
| SEQ ID NO: 6 | Z. mays PROPEP4 precursor |
| SEQ ID NO: 8 | G. max gmPEP2 mature polypeptide sequence |
| SEQ ID NO: 9 | G. max gmPEP3 polypeptide sequence fragment |
| SEQ ID NO: 10 | G. max gmPEP3 predicted mature polypeptide sequence |
| SEQ ID NO: 12 | Z. mays zmPEP2 predicted mature polypeptide sequence |
| SEQ ID NO: 13 | Z. mays zmPEP4 predicted mature polypeptide sequence |
| SEQ ID NO: 14 | A. thaliana atPEP8 predicted polypeptide sequence fragment |
| SEQ ID NO: 15 | A. thaliana atPEP8 predicted mature polypeptide sequence |
| SEQ ID NO: 16 | Brassica napus bnPEP2 predicted mature polypeptide sequence |
| SEQ ID NO: 17 | Gossypium hirsutum ghPEP1 predicted polypeptide sequence fragment |
| SEQ ID NO: 18 | G. hirsutum ghPEP1 predicted mature polypeptide sequence |
| SEQ ID NO: 19 | Oryza sativa osPEP3 predicted polypeptide sequence fragment |
| SEQ ID NO: 20 | O. sativa osPEP3 predicted mature polypeptide sequence |
| SEQ ID NO: 21 | O. sativa osPEP4 predicted polypeptide sequence fragment |
| SEQ ID NO: 22 | O. sativa osPEP4 predicted mature polypeptide sequence |
| SEQ ID NO: 23 | O. sativa osPEP5 predicted polypeptide sequence fragment |
| SEQ ID NO: 24 | O. sativa osPEP5 predicted mature polypeptide sequence |
| SEQ ID NO: 25 | O. sativa osPEP6 predicted polypeptide sequence fragment |
| SEQ ID NO: 26 | O. sativa osPEP6 predicted mature polypeptide sequence |
| SEQ ID NO: 27 | O. sativa osPEP7 predicted polypeptide sequence fragment |
| SEQ ID NO: 28 | O. sativa osPEP3 predicted mature polypeptide sequence |
| SEQ ID NO: 29 | Triticum aestivum taPEP3 predicted polypeptide sequence fragment |
| SEQ ID NO: 30 | T. aestivum taPEP3 predicted mature polypeptide sequence |
| SEQ ID NO: 48 | O. sativa osPEP3 predicted polypeptide precursor |
| SEQ ID NO: 49 | O. sativa osPEP4 predicted polypeptide precursor |
| SEQ ID NO: 50 | O. sativa osPEP5 predicted polypeptide precursor |
| SEQ ID NO: 51 | O. sativa osPEP6 predicted polypeptide precursor |
| SEQ ID NO: 52 | T. aestivum taPEP3 predicted polypeptide precursor |
| SEQ ID NO: 53 | Chimeric gmPRO1PEP2 polynucleotide sequence |
| SEQ ID NO: 54 | Chimeric ginPRO1atPEP1 polynucleotide sequence |
| SEQ ID NO: 55 | Chimeric gmPRO2PEP1 polynucleotide sequence |
| SEQ ID NO: 56 | Chimeric gmPRO2atPEP1 polynucleotide sequence |
| SEQ ID NO: 57 | Predicted chimeric gmPRO1PEP2 polypeptide sequence |
| SEQ ID NO: 58 | Predicted chimeric gmPRO1atPEP1 polypeptide sequence |
| SEQ ID NO: 59 | Predicted chimeric gmPRO2PEP1 polypeptide sequence |
| SEQ ID NO: 60 | Predicted chimeric gmPRO2atPEP1 polypeptide sequence |

The coding sequences for the new homologs and chimeric polypeptides are potential sources of transgenes to enhance yield through resistance to biotic stresses and/or abiotic stresses.

### Example 2

### Analysis of Transcription of Plant Defense Peptide Expression

Analysis of transcriptional profiles of putative plant defense peptide gene expression shows that expression of one of the corn atPROPEP1 homologs is increased in cold, suggesting a role in abiotic stress responses. The use of genes homologous to that encoding atPROPEP1 as transgenes in crops may confer biotic or abiotic stress resistance to the crop. Expression of the gene may be modified with promoters (disease-inducible, cold-inducible, drought-inducible, tissue-specific, different levels of constitutive expression, etc.) to enhance the desired phenotype, or allowing for different levels of constitutive expression, etc. to enhance the desired stress resistance phenotype.

### Example 3

### Heterologous Expression of Plant Defense Peptides

Use of a given atPROPEP gene homolog to enhance the stress resistance of a plant may also require a native peptide receptor. Alternatively, expression of an active peptide and receptor gene pair from a different plant species may be used to activate resistance to abiotic and/or biotic stresses.

### Example 4

### Use of Chimeric Genes Encoding Endogenous Plant Defense Peptides

DNA constructs were constructed for the use of the precursor portion of gmPROPEP1, or other peptide precursor, with the putative active peptide region of gmPROPEP2 or A. thaliana PEP1. The chimeric gene was, for instance, designated “gmPRO1PEP2”. SEQ ID NOs:53-56 represent gmPRO1PEP2, gmPRO1atPEP1, gmPRO2PEP1, and gmPRO2atPEP1, respectively. Thus, plant defense peptide precursor polypeptides may comprise heterologous sequences including processed sequences and mature sequences. Further, these sequences may be derived from the same species, or different species.

All of the compositions and methods disclosed and claimed herein can be made and executed without undue experimentation in light of the present disclosure. While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### REFERENCES

The following references are incorporated herein by reference:
U.S. Pat. No. 4,536,475; U.S. Pat. No. 4,693,977; U.S. Pat. No. 4,886,937; U.S. Pat. No. 4,940,838; U.S. Pat. No. 5,302,523; U.S. Pat. No. 5,322,938; U.S. Pat. No. 5,352,605; U.S. Pat. No. 5,384,253; U.S. Pat. No. 5,464,765; U.S. Pat. No. 5,501,967; U.S. Pat. No. 5,508,184; U.S. Pat. No. 5,530,196; U.S. Pat. No. 5,550,318; U.S. Pat. No. 5,591,616; U.S. Pat. No. 5,563,055; U.S. Pat. No. 5,633,435; U.S. Pat. No. 5,641,876; U.S. Pat. No. 5,780,708; U.S. Pat. No. 6,118,047; U.S. Pat. No. 6,140,078; U.S. Pat. No. 6,175,060; U.S. Pat. No. 6,252,138; U.S. Pat. No. 6,294,714; U.S. Pat. No. 6,384,301; U.S. Pat. No. 6,399,861; U.S. Pat. No. 6,403,865; U.S. Pat. No. 6,426,446; U.S. Pat. No. 6,429,357; U.S. Pat. No. 6,429,362; U.S. Pat. No. 6,437,217.
Ausubel et al., In: Current Protocols in Molecular Biology, John, Wiley & Sons, Inc, New York, 1998.
Bevan et al., Nature, 304:184-187, 1983.
Broothaerts et al., Nature, 433:629-633, 2005.
Dixon and Lamb, Annu. Rev. Plant Physiol. Plant Mol. Biol. 41:339-367, 1990.
EP 0 122 791; EP 0 120 516
Gruber et al., In: Vectors for Plant Transformation, Methods in Plant Molecular Biology and Biotechnology, Glick and Thompson (Eds.), CRC Press, Inc., Boca Raton, 89-119, 1993.
Haymes et al., In: Nucleic acid hybridization, a practical approach, IRL Press, Washington, D.C., 1985.
Herrera-Estrella et al., Nature, 303:209-213, 1983.
Horsch et al., Science, 227:1229, 1985.
Huffaker et al., Proc. Nat. Acad. Sci. USA 103:10098-10103, 2006.
Kaeppler et al., Plant Cell Rep., 8:415-418, 1990.
Klee et al., Bio/Technol., 3:637-642, 1985.
Miki et al., In: Procedures for Introducing Foreign DNA into Plants, Methods in Plant Molecular Biology and Biotechnology, Glick and Thompson (Eds.), CRC Press, Inc., Boca Raton, 67-88, 1993.
Moloney et al., Plant Cell Reports, 8:238, 1989.
Odell et al., Nature, 313:810-812, 1985.
Omirulleh et al., Plant Mol. Biol., 21:415-428, 1993.
Padgette et al., Crop Sci., 35:1451-1461, 1995.
Potrykus et al., Mol. Gen. Genet., 199:183-188, 1985.
Ryals et al., Proc. Natl. Acad. Sci. USA 92:4202-4205, 1995.
Sambrook et al., (ed.), Molecular Cloning, Cold Spring Harbor Laboratory Press, Cold Spring Harbor, N.Y., 1989.
WO06081301
Van Heeke and Schuster, J. Biol. Chem., 264:5503-5509, 1989.

1. An isolated polynucleotide comprising a sequence selected from the group consisting of:
a) a polynucleotide sequence at least 75% identical to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44 SEQ ID NO:53, SEQ ID NO:54, SEQ ID NO:55, or SEQ ID NO:56;
b) a polynucleotide encoding a polypeptide at least 85% identical to SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51, SEQ ID NO:52, SEQ ID NO:57, SEQ ID NO:58, SEQ ID NO:59, or SEQ ID NO:60; and
c) a polynucleotide that hybridizes to SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:53, SEQ ID NO:54, SEQ ID NO:55, or SEQ ID NO:56, or a complement thereof, under stringent wash conditions of 0.2×SSC at 65° for 10 minutes.

2. The isolated polynucleotide sequence of claim 1, wherein the polynucleotide comprises SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:53, SEQ ID NO:54, SEQ ID NO:55, or SEQ ID NO:56.

3. The isolated polynucleotide sequence of claim 1, wherein the polynucleotide encodes a polypeptide selected from the group consisting of SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51 SEQ ID NO:52, SEQ ID NO:57, SEQ ID NO:58, SEQ ID NO:59, and SEQ ID NO:60.

4. A construct comprising the isolated polynucleotide of claim 1 operably linked to a heterologous promoter functional in plants.

5. The isolated polynucleotide of claim 4, wherein the promoter is a stress induced promoter.

6. The isolated polynucleotide of claim 4, wherein the promoter is selected from the group consisting of a promoter induced by: osmotic stress, drought stress, cold stress, heat stress, oxidative stress, nutrient deficiency, infection by a fungus, infection by an oomycete, infection by a virus, infection by a bacterium, nematode infestation, pest infestation, weed infestation, and herbivory.

7. An isolated polypeptide sequence comprising an amino acid sequence polypeptide at least 85% identical to SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO: l 9, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:48, SEQ ID NO:49, SEQ ID NO:50, SEQ ID NO:51, SEQ ID NO:52, SEQ ID NO:57, SEQ ID NO:58, SEQ ID NO:59, or SEQ ID NO:60.

8. A composition formulated for application to a plant or a part thereof comprising the polypeptide of claim 7.

9. The composition of claim 8, formulated as a spray, a powder, a granule, or a seed treatment.

10. A method for improving the health of a plant, comprising providing to the plant the polypeptide of claim 7 in an amount that improves the health of the plant as compared to a plant of the same genotype not provided with the polypeptide.

11. The method of claim 10, wherein providing the polypeptide comprises contacting the plant with the composition of claim 8.

12. The method of claim 10, wherein providing the polypeptide comprises expressing in the plant a nucleic acid encoding the polypeptide of claim 7.

13. A transgenic plant or a part thereof transformed with the polynucleotide of claim 1.

14. The plant of claim 13, wherein the plant is selected from the group consisting of corn, soybean, cotton, canola, rice, wheat, and sunflower.

15. A part of the plant of claim 13, wherein the part comprises the polynucleotide of claim 1.

16. The part of claim 15, wherein the part is selected from the group consisting of an embryo, pollen, a cell, a root, a fruit, or a meristem.

17. A seed of the plant of claim 13, wherein the seed comprises the polynucleotide of claim 1.

18. A method of producing a plant commodity product, comprising producing the commodity product from the plant of claim 13 or a part thereof.

19. The method of claim 18, wherein the commodity product is selected from the group consisting of grain, meal, protein, flour, oil, or silage.

20. A commodity product produced by the method of claim 18, wherein the commodity product comprises the polynucleotide of claim 1.
